SEQUENCE LISTING (12 sequences)

SEQ ID NO: 1 - DNA, 10649 bp, Artificial
  Dengue virus type 4 recombinant DNA
agttgttagtctgtgtggaccgacaaggacagttccaaatcggaagcttgcttaacacagttctaacagtttgtttgaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgctgtgcttgattcccaccgtaatggcgttttccttgtcaacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcaaggaacacttcaatggctatgacgtgcatagctgttggaggaatcactctgtttctgggcttcacagttcaagcagacatgggttgtgtggtgtcatggagtgggaaagaattgaagtgtggaagcggaatttttgtggttgacaacgtgcacacttggacagaacagtacaaatttcaaccagagtccccagcgagactagcgtctgcaatattaaatgcccacaaagatggggtctgtggaattagatcaaccacgaggctggaaaatgtcatgtggaagcaaataaccaacgagctaaactatgttctctgggaaggaggacatgacctcactgtagtggctggggatgtgaagggggtgttgaccaaaggcaagagagcactcacacccccagtgagtgatctgaaatattcatggaagacatggggaaaagcaaaaatcttcaccccagaagcaagaaatagcacatttttaatagacggaccagacacctctgaatgccccaatgaacgaagagcatggaactctcttgaggtggaagactatggatttggcatgttcacgaccaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcacaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtgacggccggacagggcacatcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactgggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttaggaagcgctcttttaaagaatgatgtccctttagctggcccaatggtggcaggaggcttacttctggcggcttacgtgatgagtggtagctcagcagatctgtcactagagaaggccgccaacgtgcagtgggatgaaatggcagacataacaggctcaagcccaatcatagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactaaaaaagccgcactgtctgaaggagtgtacaggatcatgcaaagagggttattcgggaaaactcaggttggagtagggatacacatggaaggtgtatttcacacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgccatagaaccaggaaaaaatcctaaacatgtccaaacgaaacctggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctacgaactttgattttagctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagggttccaaattacaaccttatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggactttgtggtcactacagacatatctgaaatgggggccaattttagagccgggagagtgatagaccctagaagatgcctcaagccagttatcctaccagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacaaagatcgggaatggtgcttcacaggggaaagaaataaccaaattttagaagaaaacatggaggttgaaatttggactagagagggagaaaagaaaaagctaaggccaagatggttagatgcacgtgtatacgctgaccccatggctttgaaggatttcaaggagtttgccagtggaaggaagagtataactctcgacatcctaacagagattgccagtttgccaacttacctttcctctagggccaagctcgcccttgataacatagtcatgctccacacaacagaaagaggagggagggcctatcaacacgccctgaacgaacttccggagtcactggaaacactcatgcttgtagctttactaggtgctatgacagcaggcatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccatcattggtctaatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcagccgtcctaatggggcttggaaaaggatggccgctccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaacccctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtatctagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggttggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaatcattgggagaaggcttcagcgattgcaagaagagcacaaagaaacctggcattatgatcaggaaaacccatacagaacctgggcgtatcatggaagctatgaagctccttcgacaggctctgcatcctccatggtgaacggggtggtaaaactgctaacaaaaccctgggatgtgattccaatggtgactcagttagccatgacagatacaaccccttttgggcaacaaagagtgttcaaagagaaggtggataccagaacaccacaaccaaaacccggtacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgagaggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggagctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccacgctcatcaccagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcacacggccataacccaggtcaggaacctgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccccagggaggccatgcgccacggaagctgtacgcgtggcatattggactagcggttagaggagacccctcccatcactgacaaaacgcagcaaaagggggcccgaagccaggaggaagctgtactcctggtggaaggactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 2 - DNA, 10618 bp, Artificial
  Dengue virus type 4 recombinant DNA with 30 nucleotide deletion
agttgttagtctgtgtggaccgacaaggacagttccaaatcggaagcttgcttaacacagttctaacagtttgtttgaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgctgtgcttgattcccaccgtaatggcgttttccctcagcacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcgaggaacacttcaatggctatgacgtgcatagctgttggaggaatcactctgtttctgggcttcacagttcaagcagacatgggttgtgtggcgtcatggagtgggaaagaattgaagtgtggaagcggaatttttgtggttgacaacgtgcacacttggacagaacagtacaaatttcaaccagagtccccagcgagactagcgtctgcaatattaaatgcccacaaagatggggtctgtggaattagatcaaccacgaggctggaaaatgtcatgtggaagcaaataaccaacgagctaaactatgttctctgggaaggaggacatgacctcactgtagtggctggggatgtgaagggggtgttgaccaaaggcaagagagcactcacacccccagtgagtgatctgaaatattcatggaagacatggggaaaagcaaaaatcttcaccccagaagcaagaaatagcacatttttaatagacggaccagacacctctgaatgccccaatgaacgaagagcatggaactctcttgaggtggaagactatggatttggcatgttcacgaccaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcacaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtgacggccggacagggcacatcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactgggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttaggaagcgctcttttaaagaatgatgtccctttagctggcccaatggtggcaggaggcttacttctggcggcttacgtgatgagtggtagctcagcagatctgtcactagagaaggccgccaacgtgcagtgggatgaaatggcagacataacaggctcaagcccaatcatagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactaaaaaagccgcactgtctgaaggagtgtacaggatcatgcaaagagggttattcgggaaaactcaggttggagtagggatacacatggaaggtgtatttcacacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgccatagaaccaggaaaaaatcctaaacatgtccaaacgaaacctggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctacgaactttgattttagctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagggttccaaattacaaccttatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggactttgtggtcactacagacatatctgaaatgggggccaattttagagccgggagagtgatagaccctagaagatgcctcaagccagttatcctaccagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacaaagatcgggaatggtgcttcacaggggaaagaaataaccaaattttagaagaaaacatggaggttgaaatttggactagagagggagaaaagaaaaagctaaggccaagatggttagatgcacgtgtatacgctgaccccatggctttgaaggatttcaaggagtttgccagtggaaggaagagtataactctcgacatcctaacagagattgccagtttgccaacttacctttcctctagggccaagctcgcccttgataacatagtcatgctccacacaacagaaagaggagggagggcctatcaacacgccctgaacgaacttccggagtcactggaaacactcatgcttgtagctttactaggtgctatgacagcaggcatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccatcattggtctaatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcagccgtcctaatggggcttggaaaaggatggccgctccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaacccctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtatcaagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggttggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaatcattgggagaaggcttcagcgattgcaagaagagcacaaagaaacctggcattatgatcaggaaaacccatacagaacctgggcgtatcatggaagctatgaagctccttcgacaggctctgcatcctccatggtgaacggggtggtaaaactgctaacaaaaccctgggatgtgattccaatggtgactcagttagccatgacagatacaaccccttttgggcaacaaagagtgttcaaagagaaggtggataccagaacaccacaaccaaaacccggtacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgcggggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggagctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccacgctcatcaccagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcacacggccataacccaggtcaggaacctgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccccagggaggccatgcgccacggaagctgtacgcgtggcatattggactagcggttagaggagacccctcccatcactgacaaaacgcagcaaaagggggcccaagactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 3 - DNA, 1485 bp, Artificial
  Dengue virus type 4 2A with substitution at envelope protein 345 from Glu to Lys
atgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaagaaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcaaggaacacttcaatggctatgacgtgcatagctgttggaggaatcactctgtttctgggcttcacagttcaagca

SEQ ID NO: 4 - DNA, 1485 bp, Artificial
  Dengue virus type 4 2A with 30 nucleotide deletion and substitution at envelope protein 345 from Glu to Lys
atgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaagaaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcgaggaacacttcaatggctatgacgtgcatagctgttggaggaatcactctgtttctgggcttcacagttcaagca

SEQ ID NO: 5 - DNA, 1485 bp, Artificial
  Dengue virus type 4 2A with substitution at envelope protein 327 from Glu to Gly
atgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatggaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcaaggaacacttcaatggctatgacgtgcatagctgttggaggaatcactctgtttctgggcttcacagttcaagca

SEQ ID NO: 6 - DNA, 1485 bp, Artificial
  Dengue virus type 4 2A with 30 nucleotide deletion and substitution at envelope protein 327 from Glu to Gly
atgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatggaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcgaggaacacttcaatggctatgacgtgcatagctgttggaggaatcactctgtttctgggcttcacagttcaagca

SEQ ID NO: 7 - DNA, 21 bp, Artificial
  primer
tttaaggttcctcatgccaat

SEQ ID NO: 8 - DNA, 23 bp, Artificial
  primer
aaccacttttttcttgtttacat

SEQ ID NO: 9 - DNA, 23 bp, Artificial
  primer
atgtaaacaagaaaaaagtggtt

SEQ ID NO: 10 - DNA, 20 bp, Artificial
  primer
caacatgatgagggctcgta

SEQ ID NO: 11 - DNA, 20 bp, Artificial
  primer
ccagcacctccatacttgac

SEQ ID NO: 12 - DNA, 19 bp, Artificial
  primer
gtcaagtatggaggtgctg

(19) United States
(12) Patent Application Publication
(10) Pub. No.: US 2012/0269853 A1
(43) Pub. Date: Oct. 25, 2012

(54) ATTENUATED DENGUE VIRUS VACCINE CONTAINING ADAPTIVE MUTATION FROM MRC-5 CELLS

(75) Inventors: Suh-Chin Wu, Hsinchu City (TW); Hsiang-Chi Lee, Taoyuan County (TW); Hung-Ju Hsiao, Taichung City (TW); Hsiao-Han Lin, Taipei City (TW)

(73) Assignee: NATIONAL TSING HUA UNIVERSITY, Hsinchu (TW)

(21) Appl. No.: 13/090,627

(22) Filed: Apr. 20, 2011

(51) Int. Cl. A61K 39/12; A61P 31/14

(52) U.S. Cl. 424/218.1

(57) ABSTRACT

The present invention relates to an attenuated dengue virus vaccine. In present invention, target mutagenesis at Glu₃₄₅Lys was constructed in two infectious cDNA clones of a recombinant version of wild type virus DEN-4 2A and its derived 3′ NCR deletion mutant vaccine candidate virus DEN-4 2AΔ30. Using PCR-mediated site-directed mutagenesis method, the infectious cDNA clone-derived Glu₃₄₅₋Lys mutants of DEN-4 2A and DEN-4 2AΔ30 were passaged in Vero cells and MRC-5 cells for five consecutive times. The results shows that single point mutation E-Glu₃₄₅Lys of DEN-4 2A and DEN-4 2AΔ30 were found stably existed when passaged in MRC-5 cells, which means mutagenesis at Glu₃₄₅Lys of DEN-4 2A and DEN-4 2AΔ30 are both suitable to be probagated in MRC-5 cell for producing virulence attenuated dengue virus vaccine.

### FIELD OF THE INVENTION

The present invention relates to an attenuated dengue virus vaccine which comprises infectious mutants of wild type dengue virus

### BACKGROUND OF THE INVENTION

Dengue is a vector-borne virus, transmitted to humans via infected Aedes mosquitoes in tropical and sub-tropical areas. The severity of the disease varies from asymptomatic infections, to a febrile fever, or potentially life-threatening dengue hemorrhagic fever (DHF) or dengue shock syndrome (DSS). The WHO reports that two-fifths of the world's population is at risk of dengue infection, with up to 100 million cases of infections each year resulting in hundreds of thousands of cases of DHF and DSS. The virus is now endemic in more than 100 countries, affecting South-East Asia and the Western Pacific significantly, in some countries becoming the leading cause of hospitalization and death among children, with a mortality rate of up to 25,000 annually. There are dramatic increases in incidence and disease severity, attributed in part to geographic expansion of the vector by many means; the Aedes aegypti and A. albopictus mosquitoes, leading to the increased co-circulation of all dengue 1-4 serotypes in urban areas.

To date, no licensed vaccine is available. This has brought together many groups including: The Pediatric Dengue Vaccine Initiative (funded by Bill and Melinda Gates Foundation), the WHO, the US military, as well as industry and governments in many different countries to collaborate in the hopes of accelerating the development of a successful vaccine. For dengue virus, attenuation was first achieved by Sabin in 1945 by passaging the virus (DENY-1) in mouse brains (Sabin A B (1952) Research on dengue during World War II. Am J Trop Med Hyg 1(1): 30-50). However, the degree of attenuation seemed to vary depending on the strain of the virus, as many human volunteers developed a reaction in the form of a rash. This complication was addressed by Halstead and Marchette with the discovery that dengue virus could be propagated and attenuated by serial dilutions in primary dog kidney (PDK) cells (Halstead S B et al., (2003) Biologic properties of dengue viruses following serial passage in primary dog kidney cells: studies at the University of Hawaii. Am J Trop Med Hyg 69(6 Suppl): 5-11). When attenuated by serial passage in cell cultures, the molecular specifics are often unknown. Now, attenuation is often obtained by introducing genetic mutations into the genome of the virus, which interfere with the virus's ability to replicate. Recurring problems have prevented many vaccine models from advancing past clinical phases. Difficulties lie in achieving optimal attenuation of each of the four DENY serotypes, which are needed to provide a minimal level of reactogenicity and maximum immunogenicity.

### SUMMARY OF THE INVENTION

The present invention provides a dengue virus vaccine which comprises attenuated infectious mutants of wild type dengue virus, wherein the mutants have a mutation at E-E₃₄₅K site in envelope protein. The attenuated infectious mutants of wild type dengue virus mutants are selected from the group consisting of dengue virus serotype 1, dengue virus serotype 2, dengue virus serotype 3 and dengue virus serotype 4. In the preferred embodiment, the mutants comprise SEQ ID NO: 3 or SEQ ID NO: 4. In the present invention, the mutants are propagated in MRC-5 cells.

### BRIEF DESCRIPTION OF THE DRAWINGS

FIG. 1 shows experimental design for the production of MRC-5 cell adaptation mutation E-E₃₄₅K infectious cDNA clone-derived viruses.

FIG. 2 shows RNA gel analysis of DEN-4 2A, DEN-4 2A E-E₃₄₅K, DEN-4 2A E-E₃₂₇G DEN-4 2AΔ30, DEN-4 2AΔ30 E-E₃₄₅K and DEN-4 2AΔ30 E-E₃₂₇G.

FIG. 3 shows (3a) Electropherograms from the consensus sequence analysis of mutant constructs with adaptation mutations DEN-4 2A E-E₃₄₅K and DEN-4 2AΔ30 E-E₃₄₅K passaged in Vero cells and MRC-5 cells for four passages. (3b) Its parent virus infectious clone-derived DENV-4 strains DEN-4 2A and DEN-4 2AΔ30 viruses, passaged in Vero cells and MRC-5 cells for four passages. (3c) Single mutations DEN-4 2A E-E₃₂₇G and DEN-4 2AΔ30 E-E₃₂₇G passaged in Vero cells and MRC-5 cells for four passages.

FIG. 4 shows virus titers of E-E₃₄₅K and E-E₃₂₇G mutation clones in Vero cells and MRC-5 cells.

FIG. 5 shows heparin binding assay of DEN-4 2A E-E₃₄₅K, DEN-4 2AΔ30 E-E₃₄₅K, DEN-4 2A, DEN-4 2AΔ30, DEN-4 2A E-E₃₂₇G, and DEN-4 2AΔ30 E-E₃₂₇G viruses in MRC-5 cells and Vero cells.

FIG. 6 shows neurovirulence of E-E₃₄₅K and E-E₃₂₇G mutation clones in Vero cells and MRC-5 cells

FIG. 7 shows molecular modeling and surface mapping of the electrostatic field of DEN-4 E protein. Blue and red denote positive and negative charges Amino acid position 345 and 327 is shown by white arrows. Both parental and variant structures were modeled into the nuclear magnetic resonance-derived solution structure of DIII of the DEN-4 E (Molecular modeling structure is based on Protein Data Bank code 2H0P)

### DETAILED DESCRIPTION OF THE INVENTION

The present invention provides a dengue virus vaccine which comprises attenuated infectious mutants of wild type dengue virus, wherein the mutants have a mutation at E-E₃₄₅K site in envelope protein. The mutants mentioned in this specification are selected from the group consisting of dengue virus serotype 1, dengue virus serotype 2, dengue virus serotype 3 and dengue virus serotype 4. The mutants have a backbone of dengue virus serotype 4 strain 2A or dengue virus serotype 4 strain 2AΔ30, wherein prM and E genes can be replaced with dengue virus serotype 1, 2 and 3 to create mutants of all types of dengue virus. In the preferred embodiment, the mutants comprise SEQ ID NO: 3 or SEQ ID NO: 4. Furthermore, the attenuated dengue virus mutants are propagated in MRC-5 cells.

In present invention, target mutagenesis at Glu₃₄₅Lys (E₃₄₅K) in envelope protein gene was constructed in two infectious cDNA clones of a recombinant version of wild type virus DEN-4 2A and its derived 3′ NCR deletion mutant vaccine candidate virus DEN-4 2AΔ30. Using PCR-mediated site-directed mutagenesis method, the infectious cDNA clone-derived Glu₃₄₅₋Lys mutants of DEN-4 2A and DEN-4 2AΔ30 were passaged in Vero cells and MRC-5 cells for five consecutive times. Passage numbers are limited to less than 10 since live-attenuated vaccines require limited passage levels from the seed virus to prevent unsafe virus reversion. Single point mutation of E-Glu₃₄₅Lys was found to revert to Glu₃₄₅ when the virus was passaged in Vero cells. However, single point mutation E-Glu₃₄₅Lys of DEN-4 2A and DEN-4 2AΔ30 were found stably existed when passaged in MRC-5 cells. The E-Glu₃₄₅Lys substitution predicted using molecular modeling showed the increase of positive charges on the surface of E protein. The immunogenicity and virulence of these recombinant mutant viruses were further analyzed in mice. Virulence attenuation inducing by adaptation mutation implicated important information to the development of live-attenuated dengue vaccine.

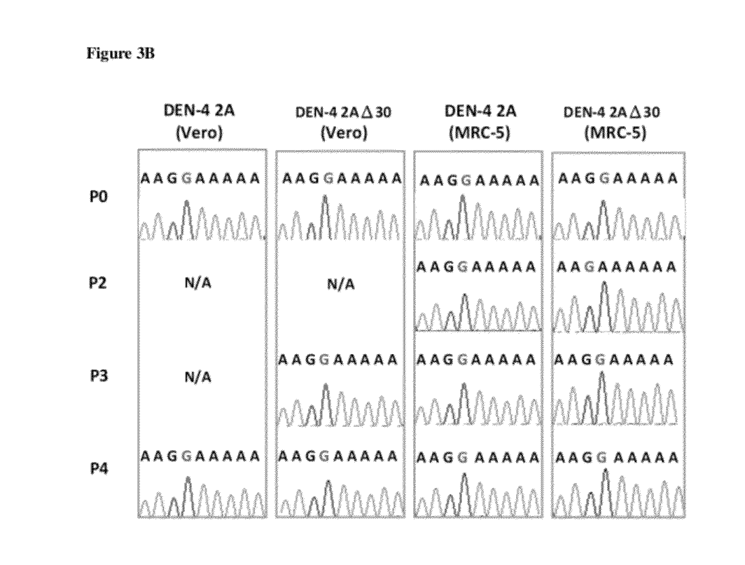

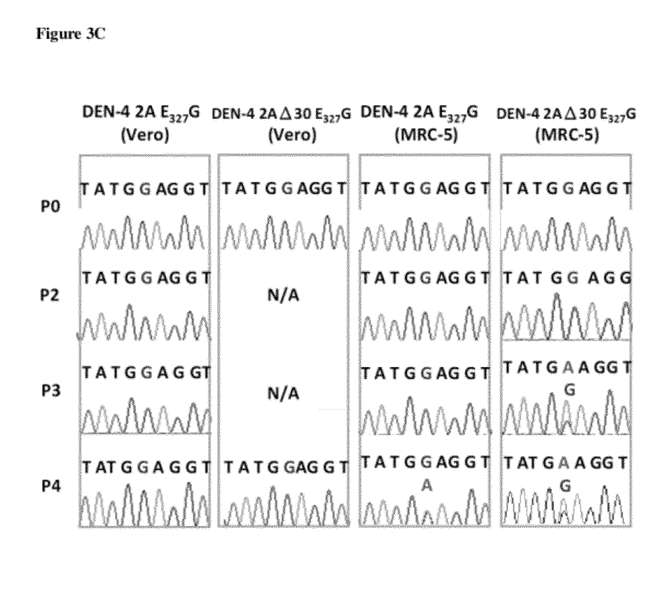

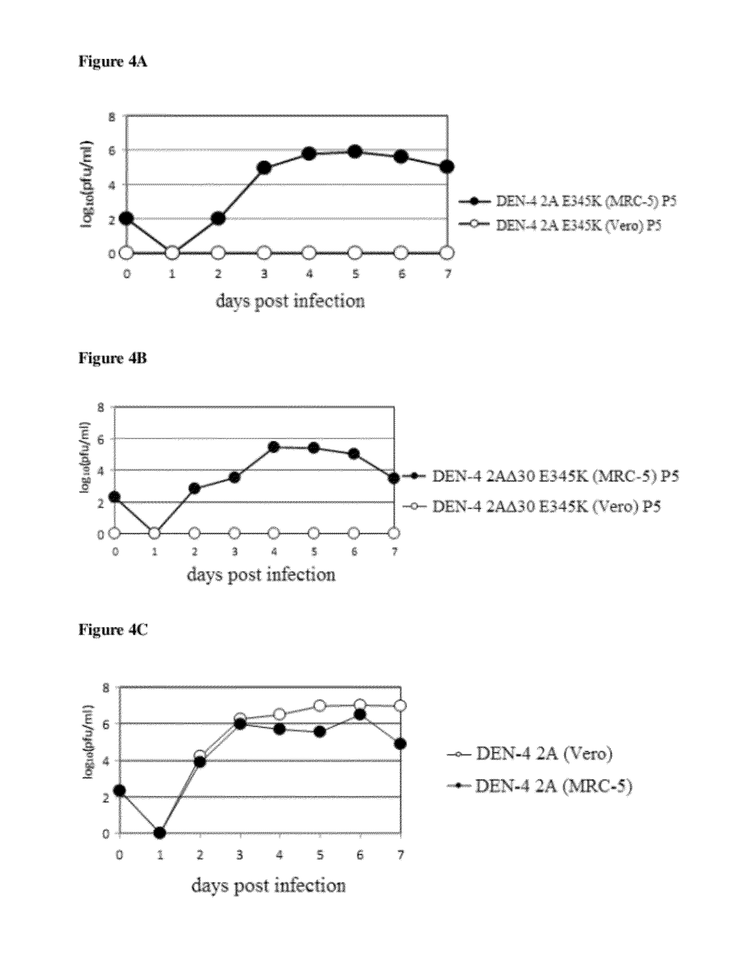

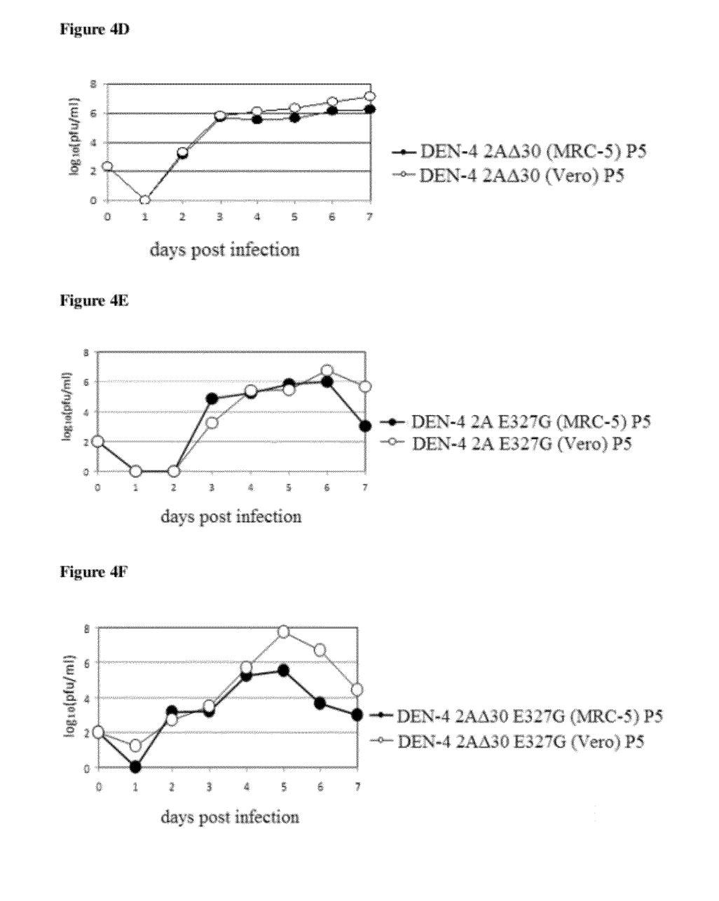

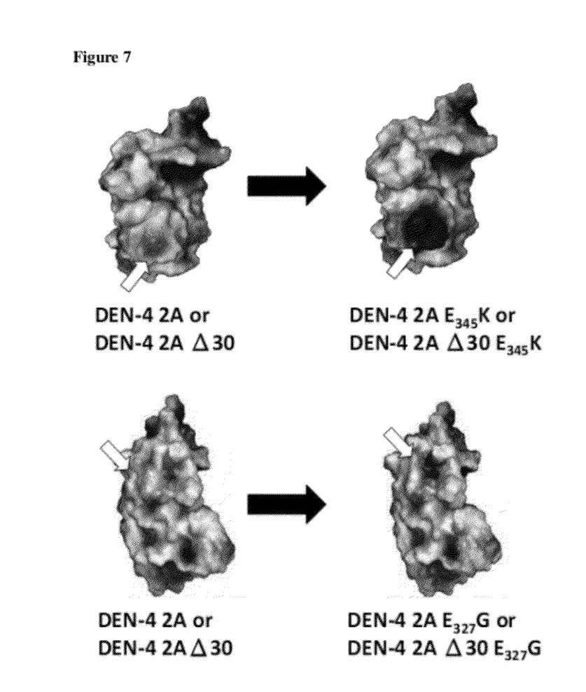

In present invention, PCR-mediated site-directed target mutagenesis technology was used to construct two infectious clones, DEN-4 2A E-E₃₄₅K and DEN-4 2AΔ30 E-E₃₄₅K, and then passaged the mutant viruses derived from these clones in Vero and MRC-5 cells for consecutive 5 passages. The E-E₃₄₅K mutation consistently presented in viruses recovered from MRC-5 cells, but it didn't present in viruses recovered from Vero cells (FIG. 3a). The analysis of virus replication patterns of DEN-4 2A E-E₃₄₅K-P5 and DEN-4 2AΔ30 E-E₃₄₅K-P5 mutant viruses in Vero and MRC-5 cells identified that both DEN-4 2A E-E₃₄₅K and DEN-4 2AΔ30 E-E₃₄₅K mutant viruses could not exist during Vero cell passage. The results of virus replication pattern also significantly indicated the E-E₃₄₅K mutation was the adaptation mutation of dengue type 4 virus vaccine candidates DEN-4 2A and DEN-4 2AΔ30 viruses in response to the MRC-5 cell environment (FIG. 4a,4b). Molecular modeling prediction and surface mapping of the electrostatic field showed that mutation of E-E₃₄₅K was predicted to increase the net positive charge at adjacent area (FIG. 7). The increase in the net positive charge resulting from E-E₃₄₅K substitution on the surface of E protein domain III was considered to be accompanied with the creation of a new binding site for heparin sulfate (HS), wherein the HS-binding ability is relative to the reduction of virulence of dengue viruses.

### Materials and Methods

### Cells and Media

Vero, Vero E6, and MRC-5 cells were all obtained from the Bioresource Collection and Research Center (BCRC) of the Food Industrial Research and Development Institute, Hsinchu, Taiwan. Vero cells (African green monkey kidney cells) were derived from ATCC CCL-81, and its BCRC number is 60013. Vero E6 cells (African green monkey kidney cells) were a clone of VERO 76 cells (ATCC CRL-1587) and were cloned by the dilution method into microtiter plates in 1979 by P. J. Price. The BCRC number of Vero E6 cells is 60476 (derived from ATCC CRL-1586). Vero E6 cells are more sensitive to several hemorrhagic fever viruses. MRC-5 cells (human embryonal lung fibroblasts) were derived from ATCC CCL-171, and its BCRC number is 60023. Vero, Vero E6, and MRC-5 cells were grown in Dulbecco's Modified Essential Medium (DMEM) (Invitrogen) supplemented with 10% heat-inactivated fetal bovine serum (FBS) and 100 U/ml of penicillin G sodium-streptomycin (Invitrogen).

### Viruses

Stock viruses were prepared from supernatants of infected C6/36 cells grown in Hank's MEM medium (Gibco-BRL) plus supplements 6 days post-infection at 28° C. The plasmids of DEN-4 2A and its 3′NCR deletion mutant DEN-4 2AΔ30 contained the full-length genomic sequences. The plasmids were first linearized by cleavage with restriction enzyme Kpn I and then added to a transcription reaction mixture (Promega kit) containing m⁷G(5′)ppp(5′)G (Merck) for cap addition at the RNA 5′-end. After incubation at 37° C. for 1.5 hour, the RNA product was purified with TRIzol reagent (Invitrogen) according to manufacturer's instructions. Prior to RNA transfection, subconfluent Vero cells and MRC-5 cells in a 6-well plate were rinsed once with serum-free medium and then covered with 0.3 ml of DMEM medium per well. The transfection mixture was prepared by adding 4 μl of DMRIE-C reagent (Invitrogen) to 1 ml of DMEM, then mixing with 10 μg of the RNA product. The transfection mixture was added directly to cell monolayer. After 18 hours incubation at 37° C., either DMEM with 10% FBS or M-VSFM medium was added to the well. Eight days after transfection, culture supernatants were collected. All virus stocks were stored at −80° C. freezer for further analysis. The virus titer was then determined by plaque assay on a Vero-E6 cell line. To prepare high titers of DENY, virus supernatant was concentrated with a Centriplus device (10-kDa cutoff) (Amicon; Millipore) by 2,800 rpm centrifugation for 30 minutes before the plaque assay. The virus titer could reach 10⁹ PFU/ml after concentration. The inoculum was prepared by diluting virus stocks in phosphate-buffered saline (PBS) immediately before inoculation. In order to confirm the homogenous virus population for our study, biological clones of each passage regimens were generated by one round of plaque purification in Vero cells or MRC-5 cells. Medium 199 (Gibco) containing 3% FBS was used for plaque purifications done in six-well culture plates by the agarose overlay method with neutral red staining described previously. The agarose plug was carefully removed without disturbing the monolayer. Each of the selected clones was then propagated once in Vero cells or MRC-5 cells in order to confirm the viability of each clone.

Target Mutagenesis, Construction of DEN-4 2A E₃₄₅K, DEN-4 2AΔ30 E₃₄₅K, DEN-4 2A E₃₂₇G and DEN-4 2AΔ30 E₃₂₇G Infectious cDNA Clones, and Recovery of Mutant Viruses

The clone-derived virus, DEN-4 2A and DEN-4 2AΔ30, exhibit the same phenotypes as the DENV-4 vaccine candidate strain 814669 virus and its less virulent 3′ NCR deletion mutant virus and was used as wt control. Target mutagenesis generating the mutant cDNA clones were performed by using overlapped PCR method. To construct DEN-4 2A E₃₄₅K, DEN-4 2AΔ30 E₃₄₅K, DEN-4 2A E₃₂₇G and DEN-4 2AΔ30 E₃₂₇G infectious cDNA clones, PCR fragments containing corresponding mutations were amplified by two rounds of PCR reactions. The first round was done by using primer pairs NsiI-f: 5′-TTTAAGGTTCCTCATGCCAAT-3′ (SEQ ID NO:7) and E₃₄₅K-r: 5′-AACCACTTTTTTCTTGTTTACAT-3′ (SEQ ID NO:8), and E₃₄₅K-f: 5′-ATGTAAACAAGAAAAAA (this changed E gene amino acid no. 345 from Glu to Lys; from GAA to AAA) GTGGTT-3′ (SEQ ID NO:9) and StuI-r 5′-CAACATGATGAGGGCTCGTA-3′ (SEQ ID NO:10) for construction of DEN-4 2A E₃₄₅K and DEN-4 2AΔ30 E₃₄₅K; primer pairs NsiI-f 5′-TTTAAGGTTCCTCATGCCAAT-3′ (SEQ ID NO:7) and E₃₂₇G-r: 5′-CCAGCACCTCCATACTTGAC-3′ (SEQ ID NO: 11), and E₃₂₇G-f: 5′-GTCAAGTATGGA (this changed E gene amino acid no. 327 from Glu to Gly; from GAA to GGA) GGTGCTG-3′ (SEQ ID NO:12) and StuI-r 5′-CAACATGATGAGGGCTCGTA-3′ (SEQ ID NO:10) for construction of DEN-4 2A E₃₂₇G and DEN-4 2AΔ30 E₃₂₇G. The infectious cDNA clones of parental viruses DEN-4 2A and DEN-4 2AΔ30 were used as templates in PCR reactions. The second round was done by using the same primer pairs NsiI-f: 5′-TTTAAGGTTCCTCATGCCAAT-3′ and StuI-r 5′-CAACATGATGAGGGCTCGTA-3′. The 2,003 bp NsiI-StuI PCR fragments were cloned into the pJET1.2/blunt cloning vector (Fermentas Life Sciences Corp.) for amplification. To introduce changes at E protein residues 345 or 327, a 2 kb region flanked by NsiI and StuI restriction enzyme sites in DEN-4 2A and DEN-4 2AΔ30 infectious clones were replaced with NsiI-StuI fragments derived from confirmed clones, which contained E₃₄₅K or E₃₂₇G mutations. The mutant DEN-4 2A E₃₄₅K, DEN-4 2AΔ30 E₃₄₅K, DEN-4 2A E₃₂₇G and DEN-4 2AΔ30 E₃₂₇G infectious clones were confirmed by sequencing analysis. The mutant plasmids were first linearized by cleavage with restriction enzyme KpnI and then added to a transcription reaction mixture, transcribed using SP6 RNA polymerase within the RiboMAX™ large scale RNA production system (Promega Corp.). Full-length RNA transcripts were further capped with m⁷G(5′)ppp(5′)G at the RNA 5′-end by using Script Cap Capping enzyme (EPICENTRE Corp.). After incubation at 37° C. for 1 hour, the RNA product was purified with TRIzol LS reagent (Invitrogen Corp.) according to manufacturer's instructions. Prior to RNA transfection, subconfluent Vero cells and MRC-5 cells in a 6-well plate were rinsed once with serum-free medium and then covered with 0.3 ml of DMEM medium per well. The transfection mixture was prepared by adding 4 n1 of DMRIE-C reagent (Invitrogen) to 1 ml of DMEM, then mixing with 10 μg of the RNA product. The transfection mixture was added directly to cell monolayer. After 18 hours incubation at 37° C., either DMEM+10% FBS or M-VSFM medium were added to the well. Eight days after transfection, culture supernatants were collected. All virus stocks were stored at −80° C. freezer for further analysis. The virus titer was then determined by plaque assay on a Vero-E6 cell line. To prepare high titers of DENY, virus supernatant was concentrated with a Centriplus device (10-kDa cutoff) (Amicon; Millipore) by centrifugation before the plaque assay. The virus titer could reach 10⁹ PFU/ml after concentration. The inoculum was prepared by diluting virus stocks in Hank's balanced salt solution (Invitrogen) containing 0.4% bovine serum albumin fraction V (Gibco) (HBSS-0.4% BSA fraction V) immediately before inoculation.

### Determination of Cell Density and Virus Titer

The number of cells attached to the microcarriers was determined by nuclei staining. Briefly, a 1-ml sample of the microcarrier culture was taken and centrifuged at 200 g for 5 min to remove the supernatant. The pellets were treated with 1 ml 0.1 M citric acid [containing 0.1% (w/v) crystal violet] and incubated at 37° C. for 1 hour. The released nuclei were counted in a hemocytometer. The virus titer was measured by 10-fold serial dilutions of the culture supernatant in duplicate infections of Vero-E6 cell monolayers in a 6-well plate. After 1 hour incubation at 37° C., 4 ml of medium containing 1× Eagle's Minimum Essential Medium (EMEM) (Invitrogen), 1.1% methylcellulose, and 100 U/ml of penicillin G sodium-Streptomycin (4 ml/well) was added to each well. Virus plaques were stained with 1% crystal violet dye six days after incubation. The infectivity titer in plaque forming units (PFU) per ml was determined

### Sequencing DEN-4 Fragments

DNA fragments were synthesized from DEN-4 RNA by RT-PCR using Platinum® Pfx DNA polymerase with forward and reverse primers WE1 and W02R. The DNA products were purified by Gel/PCR DNA fragments extraction kit (Geneaid, Taiwan). The nucleotide sequences of each fragment were determined by Mission Biotech Inc., Taipei, Taiwan. Sequences were aligned in the WE1/W02R region (1,653-bp) using the program Lasergene version 6.00 to generate the consensus sequence for each of the three fragments. The mean diversity of amino acids was determined as the number of amino acid substitutions divided by the total number amino acids sequenced.

### Surface Mapping of Electrostatic Field

Molecular modeling showed the mutations of Glu₃₄₅Lys and Glu₃₂₇Gly which were predicted by SWISS-MODEL based on the DEN-4 E DIII structural model determined by the nuclear magnetic resonance (NMR) spectroscopic method (Protein Data Bank code 2H0P). Surface mapping of the electrostatic field of DENV-4 E DIII was display using the software PyMOL (version 0.99, Delano Scientific). Blue and red colors denote positive and negative charges.

### Heparin-Sepharose Binding Assay

Heparin-Sepharose and control protein A-Sepharose beads (Pharmacia, Uppsala, Sweden) were suspended in phosphate-buffered saline (30%, wt/vol) and equilibrated before use by pelleting and washing three times in HBSS-BSA plus 10 mM HEPES (pH 8.0). 10⁵ PFU parental DEN-4 2A, DEN-4 2AΔ30, DEN-4 2A harboring a E Glu₃₄₅-Lys mutation (DEN-4 E₃₄₅K), DEN-4 2AΔ30 harboring a E Glu₃₄₅-Lys mutation (DEN-4 2AΔ30 E₃₄₅K), DEN-4 harboring a Glu₃₂₇-Gly mutation (DEN-4 E₃₂₇G), and DEN-4 2AΔ30 harboring a Glu₃₂₇-Gly mutation (DEN-4 2AΔ30 E₃₂₇G) viruses diluted in 100 μl Hank's balanced salt solution (Invitrogen) containing 0.2% bovine serum albumin (Gibco) (HBSS-BSA) plus 100 μl of HBSS-BSA with or without Sepharose beads were mixed in Eppendorf tubes and held at 4° C. for 6 h with repeated mixing. Virus-bead mixtures were then centrifuged for 5 min at 6,000×g at 4° C. to pellet the Sepharose beads, and infectious titers in supernatants were determined by focus-forming assay (6) on Vero cells or MRC-5 cells.

### Cell Binding Inhibition ELISA

To determine the binding ability of clone-derived mutated viruses, serial dilution of the virus samples were directly incubated with Vero cells. Confluent monolayers of Vero cells in 96-well plates were rinsed with PBS and then fixed by adding 10% formaldehyde overnight prior to blocking with 5% skim milk in TBST. The mutated viruses were incubated with fixed Vero cells monolayers for 1 h at room temperature. After being washed with TBST, bound viruses were quantified by ELISA analysis with mAb HB-114. The bound antibodies were detected after incubation with the anti-mouse IgG conjugated to peroxidase (KPL) for 1 h at room temperature. The ELISA products were developed with a chromogen solution containing ABTS and hydrogen peroxide and then the A₄₀₅ was measured. For binding inhibition assays by GAGs, each mutated viruses were preincubated with HBSS, and GAGs, including heparin, heparin sulfate, chondroitin sulfates A, B, and C, and hyaluronic acid (Sigma Chemical Co., St. Louis, Mo.), at room temperature for 1 h. The GAG-virus mixture was added into fixed Vero cell monolayers for additional 1 h incubation at room temperature. After being washed with TBST, bound viruses were quantified by ELISA analysis.

### Mouse Studies

Neutralizing antibody responses were tested in 3-week-old ICR mice (colony maintained at BioLASCO Taiwan CO., Ltd.). They were inoculated intraperitoneally with 250 μl of diluent HBSS-0.4% BSA fraction V (as mock) or diluent containing 10⁴ PFU of viruses and were boosted with the same amount of viruses 3 weeks later. Mice were bled 2 days prior to the boost and 3 weeks after boosting.

### Neutralization Assays

Mouse serum samples were tested for neutralizing antibodies by serum dilution-plaque reduction neutralization test (PRNT) without addition of complement. Seventy-five PFU of virus was incubated with equal volumes of serial twofold dilutions of heat-inactivated (56° C. for 30 min) mouse serum specimens overnight at 4° C. Six-well plates of Vero cells were inoculated with the serum-virus mixtures and incubated at 37° C. in a 5% CO₂ incubator for 1.5 h. Plates were then treated as described for the plaque titration protocol. Back titrations of the input DEN-4 2A and DEN-4 2AΔ30 virus were included in quadruplicate in each assay. The neutralizing antibody titer was identified as the highest serum dilution that reduced the number of virus plaques in the test by 50% or greater. The 50% neutralization inhibition dose (ID₅₀) that is the geometric reciprocal of the serum dilution yielding 50% reduction in the virus titer was obtained using the software, ID50 version 5.0 (John L. Spouge, National Center for Biotechnology Information, Bethesda, Md., USA).

### Neurovirulence in Suckling Mice

Litters of newborn (less than 1 day old) outbred white ICR mice (BioLASCO Taiwan Co., Ltd) were inoculated intracranially with 30 μl of diluent (Mock) as diluent or diluent containing 10⁴ PFU of DEN-4 2A E₃₄₅K (MRC-5)-P4, DEN-4 2A (Vero)-P4, DEN-4 2A E₃₂₇G (Vero)-P4, DEN-4 2AΔ30 E₃₄₅K (MRC-5)-P4, DEN-4 2AΔ30 (Vero)-P4 and DEN-4 2AΔ30 E₃₂₇G (MRC-5)-P4. The diluent was HBSS-0.4% BSA fraction V (GIBCO, U.S.A.). They were observed daily for 18 days and the survival rate of each experimental group was evidenced by moribund status, paralysis, or death.

The examples below are non-limiting and are merely representative of various aspects and features of the present invention.

### EXAMPLE

### Example 1

### Mutations of E-Glu₃₄₅Lys and E-Glu₃₂₇Gly in DEN-4 2A and DEN-4 2AΔ30 Infectious cDNA Clones

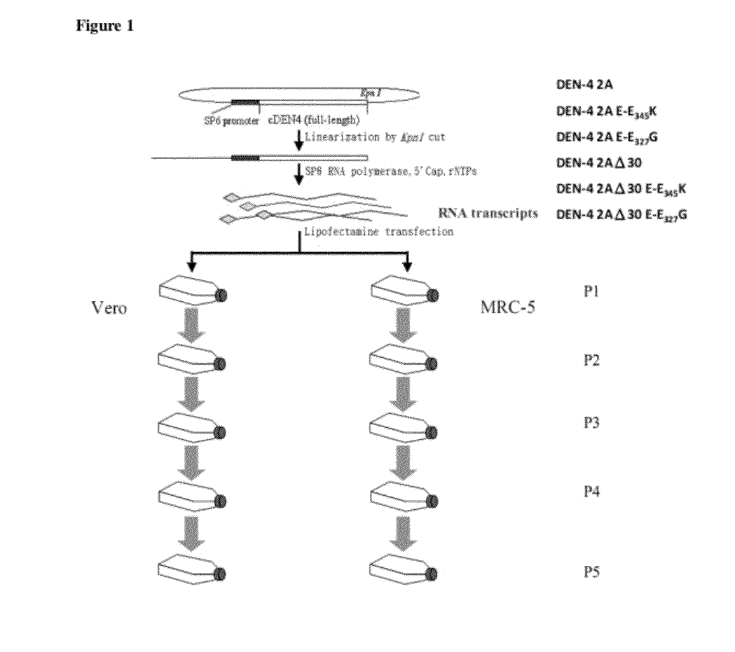

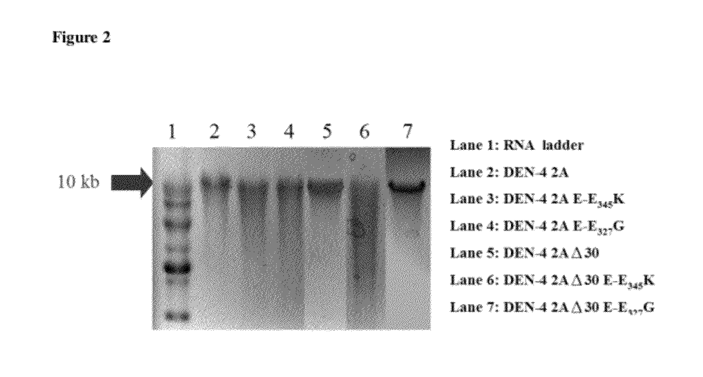

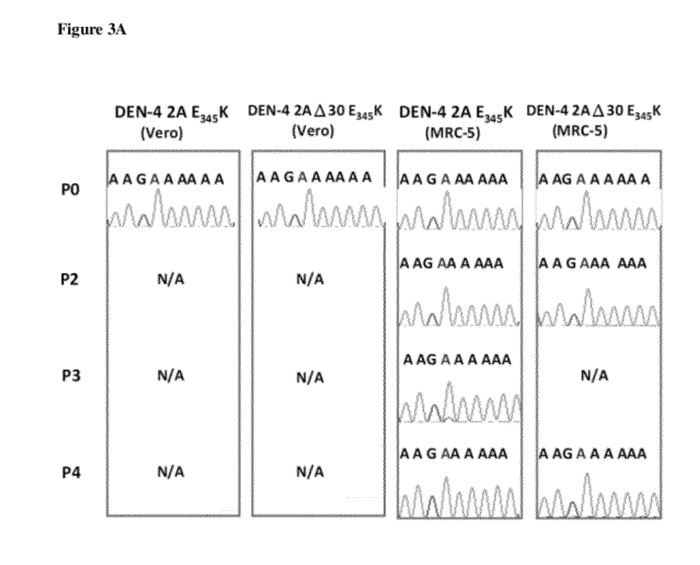

Target mutagenesis of E-Glu₃₄₅Lys (E-E₃₄₅K) and E-Glu₃₂₇Gly (E-E₃₂₇G) on two infectious cDNA clones, DEN-4 2A and DEN-4 2AΔ30 was conducted. RNA transcripts were obtained by incubating the cDNAs with SP6 RNA polymerase and rNTPs for 2 h and then the in vitro transcribed RNAs were capped with GTP and 5′-Cap capping enzyme for 1 h at 37° C. (FIG. 1). The in vitro RNA transcripts (P0) were around 11 kb as single bands analyzed by RNA gel electrophoresis (FIG. 2). The RNA transcripts were then transfected into Vero cells and MRC-5 cells individually, and propagated in Vero cells and MRC-5 cells for five consecutive passages (P1, P2, P3, P4, P5) (FIG. 1). The virus stocks obtained from each passage in Vero cells or MRC-5 cells were extracted and RT-PCR for sequencing analysis. The electropherograms show that the mutations of E-E₃₄₅K of the DEN-4 2A and DEN-4 2AΔ30 clones were not detectable in Vero cells at P2, P3, and P4. However, the mutations of E-E₃₄₅K were consistent in MRC-5 cells (FIG. 3a). In contrast, the consensus (wild type) sequences of DEN-4 2A and DEN-4 2AΔ30 clones were steady in Vero cells and MRC-5 cells at P0, P2, P3, and P4 (FIG. 3b). The mutations of E-E₃₂₇G of DEN-4 2A and DEN-4 2AΔ30 clones were consistent in Vero at P0, P2, P3, and P4. However, the mutations of E-E₃₂₇G clones propagated in MRC-5 cells, appeared as a mixture of G and A nucleotides at P4 of the DEN-4 2A clone and at P3 and P4 of the DEN-4 2AΔ30 clone (FIG. 3c).

### Example 2

### Replication Kinetics of E-Glu₃₄₅Lys and E-Glu₃₂₇Gly Mutation Clones in Vero Cells and MRC-5 Cells

The E-E₃₄₅K and E-E₃₂₇G mutant viruses derived from DEN-4 2A and DEN-4 2AΔ30 clones were investigated in Vero cells and MRC-5 cells. The E-E₃₄₅K mutant virus derived from the DEN-4 2A clone was only able to grow in MRC-5 cells. No virus titer was detected in the E-E₃₄₅K mutant virus in Vero cells (FIG. 4). However, the wild type and the E-E₃₂₇G mutant viruses derived from the DEN-4 2A clone were able to grow in both Vero cells and MRC-5 cells (FIG. 4c-4f). The maximum titers of the wild-type and E-E₃₂₇G mutant viruses propagated in Vero cells were slightly higher as compared to these viruses propagated in MRC-5 cells.

### Example 3

### Neurovirulence of E-Glu₃₄₅Lys and E-Glu₃₂₇Gly Mutation Clones in Vero Cells and MRC-5 Cells

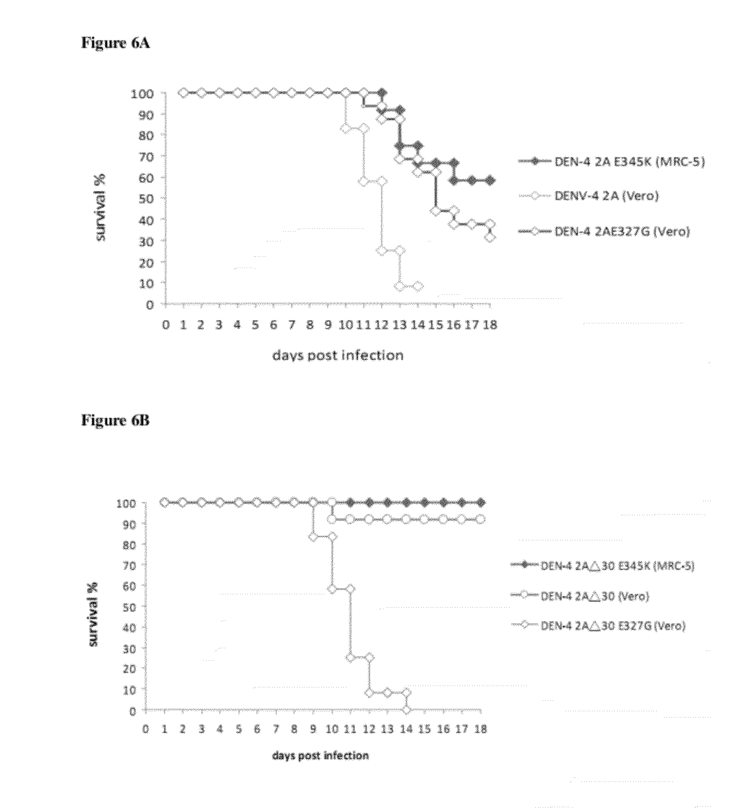

Since the virus genome may change during cell passages through adaptive selection, and mutations may affect cell tropism and virus virulence. The neurovirulence of the E-E₃₄₅K and E-E₃₂₇G mutant viruses derived from DEN-4 2A and DEN-4 2AΔ30 clones was further investigated. The virulence of these recombinant mutant viruses were analyzed in newborn ICR mice. The E-E₃₄₅K mutant virus derived from the DEN-4 2A clone showed less virulent compared to its wild type DEN-4 2A virus and E-E₃₂₇G mutant virus in newborn ICR mice. However, DEN-4 2A E-E₃₄₅K mutant virus still killed 41.7% of the mice, compared to DEN-4 2A E-E₃₂₇G mutant virus and DEN-4 2A virus killed 68.8% and 92% of the mice, respectively (FIG. 6a). The average survival times of DEN-4 2A E-E₃₄₅K, DEN-4 2A E-E₃₂₇G, and DEN-4 2A viruses infected mice were 15.91±2.96, 14.62±2.84, and 8.42±1.21 days, respectively. The E-E₃₄₅K mutant virus derived from the DEN-4 2AΔ30 clone showed avirulent compared to its wild type DEN-4 2AΔ30 virus and E-E₃₂₇G mutant virus in newborn ICR mice. DEN-4 2AΔ30 E-E₃₄₅K mutant virus killed 0% of the mice, compared to DEN-4 2AΔ30 E-E₃₂₇G mutant virus and DEN-4 2AΔ30 virus killed 100% and 8.4% of the mice, respectively (FIG. 6b). The average survival times of DEN-4 2AΔ30 E-E₃₄₅K, DEN-4 2AΔ30 E-E₃₂₇G, and DEN-4 2AΔ30 viruses infected mice were >18, 10.72±1.42, and 17.33±2.31 days, respectively.

### Example 4

### Mutations of E-Glu₃₄₅Lys and E-Glu₃₂₇Gly Increased Heparin Binding

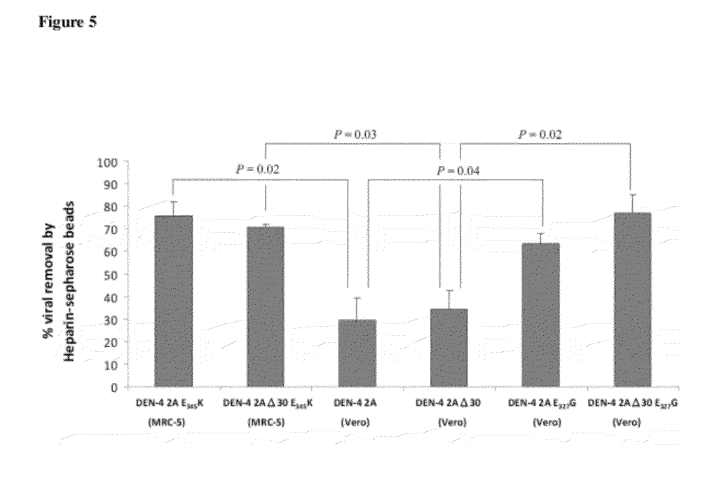

Based on the DEN-4 structural model determined by nuclear magnetic resonance (NMR) spectroscopic method (Protein Data Bank code 2H0P) (Volk D E et al., (2007) Solution structure of the envelope protein domain III of dengue-4 virus. Virology 364:147-154), Glu₃₄₅ in DEN-4 E is located within the C loop at the lower central region of DIII and is nearby on the virion surface. Glu₃₂₇ in DEN-4 E is located within the BC loop at the upper lateral ridge of DIII and is accessible on the virion surface, based on the DEN-2 structural model determined by cryo-electron microscopy (cryo-EM) (Protein Data Bank code 1THD). Molecular modeling predicted showed that both mutations of E-Glu₃₄₅Lys and E-Glu₃₂₇Gly are predicted to increase the net positive charge at the local area, as shown by surface mapping of the electrostatic field generated by the PyMOL software (version 0.99, Delano Scientific, CA, USA) using the DEN-4 DIII NMR reconstruction. The transition of the mutation E-Glu₃₄₅Lys showed more net positive charge generated than that of the mutation of E-Glu₃₂₇Gly. Heparin binding assay was carried to examine the binding of the parent and both DEN-4 mutated viruses to heparin-Sepharose beads. The results showed that the fraction of the mutant containing either the E-Glu₃₄₅Lys and E-Glu₃₂₇Gly retained by heparin beads was significantly higher than that of the parental DEN-4 2A and its 3′UTR 30 nucleotides deletion derivative DEN-4 2AΔ30 viruses (FIG. 5). The mutation of DEN-4 2A Glu₃₄₅Lys appeared to show a higher level of binding to heparin than the mutation of DEN-4 2A Glu₃₂₇Gly (75.71±6.06% versus 63.57±4.39%). However, the mutation of DEN-4 2AΔ30 Glu₃₄₅Lys appeared to show an approximate level of binding to heparin than the mutation of DEN-4 2AΔ30 Glu₃₂₇Gly (70.71±1.01% versus 76.82±8.4%).

What is claimed is:

1. A dengue virus vaccine comprising attenuated infectious mutants of wild type dengue virus, wherein the mutants have a mutation at E-E₃₄₅K site in envelope protein.

2. The dengue virus vaccine of claim 1, wherein the mutants are selected from the group consisting of dengue virus serotype 1, dengue virus serotype 2, dengue virus serotype 3 and dengue virus serotype 4.

3. The dengue virus vaccine of claim 1, wherein the mutants are propagated in MRC-5 cells.

4. The dengue virus vaccine of claim 1, wherein the mutants comprising SEQ ID NO: 3 or SEQ ID NO: 4.

5. The dengue virus vaccine of claim 1, wherein the mutant is SEQ ID NO: 3.

6. The dengue virus vaccine of claim 1, wherein the mutant is SEQ ID NO: 4.
